(19) United States
(12) Patent Application Publication
(10) Pub. No.: US 2024/0336890 A1
(43) Pub. Date: Oct. 10, 2024

(54) METHOD FOR THE PRODUCTION OF A SHAPE-MEMORY TISSUE AND RELATIVE USES

(71) Applicants: UNIVERSITA’ DEGLI STUDI DI PAVIA, Pavia (IT); FONDAZIONE IRCCS POLICLINICO SAN MATTEO, Pavia (IT)

(72) Inventors: Ida GENTA, Pavia (IT); Bice CONTI, Torre d Isola (PV) (IT); Rossella DORATI, Pavia (IT); Marco BENAZZO, Pavia (IT); Silvia PISANI, Finale Ligure (SV) (IT); Maria Antonietta AVANZINI, Pavia (IT)

(21) Appl. No.: 18/578,773

(22) PCT Filed: Jul. 19, 2022
(86) PCT No.: PCT/IB2022/056647

§ 371 Date: Jan. 12, 2024

(30) Foreign Application Priority Data
Jul. 20, 2021 (IT) 102021000019256

(51) Int. Cl. C12N 5/00; C12N 5/0775; D01D 5/00; D01F 6/62; D01F 6/84; D01F 6/92

(52) U.S. Cl. CPC C12N 5/0062; C12N 5/0662; D01D 5/0038; D01F 6/625; D01F 6/84; D01F 6/92; C12N 2513/00; C12N 2533/40; D10B 2331/041; D10B 2509/00

(57) ABSTRACT

The present invention consists of a method for the production of a shape-memory tissue through the use of polymeric matrices capable of temporarily modifying their shape, following the application of an external stimulus, and at the same time capable of supporting the vehiculation of cells and/or drugs.

The present invention consists of a method for the production of a shape-memory tissue through the use of polymeric matrices capable of temporarily modifying their shape, following the application of an external stimulus, and at the same time capable of supporting the vehiculation of cells and/or drugs.

There are various examples in literature of polymeric matrices having shape-memory properties for the most varied biomedical applications.

In 2018, Tatu and colleagues [1] published an article relating to the use of a physical mixture of polylactic acid (PLA) and polycaprolactone (PCL) for the preparation of a self-expanding film to be used for the in situ treatment of spina bifida in the fetus. The formulation that showed the best self-expansion capacity was that with a glass transition temperature (Tg) of around 37.6±1.28° C. The polymeric film proposed by Tatu and colleagues however is obtained from a physical mixture of the two polymers, therefore the percentage composition of the respective polymers in the different areas of the final product may not be constant and difficult to reproduce. Furthermore, the presence of cells or drugs is not provided for.

In 2014 [2], Bao and collaborators proposed electrospun matrices based on polylactate-co-trimethylene carbonate with shape-memory properties for bone regeneration. The electrospun matrices proposed were subjected to heat treatment to make them acquire an extended shape starting from an original spiral shape. Also in this case cellularization is not provided for. Bao and collaborators use only cells for evaluating the biocompatibility of their product, but the cells are not a component of the finished product.

In 2018, Zhao and colleagues produced polymeric matrices based on PCL and methacrylate gelatin using the co-electrospinning technique.

Starting from tubular matrices, as they were deposited on a cylindrical collector, the matrices were brought to an extended conformation by induction. The subsequent increase in temperature to values of 37° C. led to the recovery of the original tubular shape. The programmed shape-memory matrices presented by Zhao were used as membranes for obtaining 3D endothelialization models. Once the flat/stretched shape had been induced, the matrices were cellularized and then brought back to the tubular conformation by increasing the temperature. Cellularization therefore takes place as an intermediate step in the process for inducing the transitory form and recovering the original configuration.

Tseng and colleagues [4] proposed thermo-responsive electrospun matrices capable of changing orientation at the level of their microscopic architecture. The effect given by this change of direction was evaluated in terms of the different cell-differentiation response by mesenchymal cells. The electrospun polyurethane-based matrices (glass transition temperature 48-49° C.), once heated above the glass transition temperature, were mechanically subjected to extensive stress in order to induce a preferential orientation of the same. The preferential alignment induced is maintained in order to study the effect on cell differentiation. Once the mechanical stress has been removed, the fibers return to their original random/disordered structure. The work does not evaluate the shape variation of the matrices at a macroscopic level, but only at a microscopic level purely for studying the interaction of the architecture of the support with cell growth. Furthermore, this product refers to the use of a polymer, polyurethane, whose shape-transition properties occur at an excessively high temperature (48-49° C.) to be applied in a physiological environment in the human body.

The authors of the present invention have now developed a cellularized polymeric matrix and/or charged with an active principle (e.g. a drug, a hormone, a growth factor or a biologically derived product) characterized by a unique, homogeneous and reproducible composition. extremely versatile for use in the medical field, for example in tissue regeneration.

A technical problem that the invention proposes to solve is, in fact, the implantation of polymeric matrices for tissue regeneration, and/or systems for the delivery of drugs, in sites of the human body that are difficult to reach (such as, for example, the prostate or the spina bifida).

A further technical problem that the present invention proposes to reduce is the invasiveness of some surgical operations that involve replacement implants of portions of tissue/organs (i.e. esophagus, trachea). By way of non-limiting example, the polymeric matrices according to the invention find application in minimally invasive robotic surgery to obtain targeted therapy at the intra-pleural, thoracic, laryngological-esophageal, intestinal, arterial-venous, cardiac, intra-prostatic, intra-uterine, intra-articular, ocular level.

The advantage offered by the present invention is the possibility of obtaining an engineered shape-memory polymer matrix, which can be implanted in human beings with a minimally invasive technique as the reduced volumetric dimensions of the rolled matrix in the transitory configuration allow for an easier implantation so as to reduce incisions, the healing time of the wound, associated pain and risk of infection. The subsequent unrolling of the matrix, induced by an external stimulus, preferably thermal and of a physiological entity (i.e. which falls within the normal parameters of body temperature) after implantation in the site of action, will allow the matrix to exert the required action which may be cell vehiculation or drug delivery on site.

The invention proposed also proves to be useful in overcoming the limitations of local cell therapy as the injection or deposition in situ of the cells alone does not guarantee their permanence for the time necessary for correct repopulation, obtaining partial results. With this engineered product, cells can be conveyed locally in order to ensure a correct and long-lasting positioning, so as to be able to exert a suitable and timely action for the healing process.

As anticipated above, although various scientific papers are present in literature in which the shape-memory property of polymeric matrices is exploited, none of them have provided for the cellularization step of the polymeric support upstream of the shape-changing steps. In the method according to the invention, in fact, the whole induction process of the transitory form, its maintenance and the recovery of the original configuration are effected with previously cellularized matrices. The same principle can be applied to the use of these engineered matrices carrying an active ingredient with a pharmacological action and/or a biological derivative. Instead of systemically administering an active ingredient that can potentially lead to adverse effects in non-target organs, in situ administration via controlled release from the polymeric matrix can favour specific therapeutic activity by limiting the systemic toxicity of drugs.

The present invention therefore relates to a shape-memory polymeric matrix that can be obtained by means of the following steps:
a) electrospinning of a PLA-PLC copolymer or PLA and PLC polymers in a physical mixture in which the PLA: PCL weight ratio ranges from 80:20 to 60:40, preferably 70:30.
b) sanitation or sterilization of the polymer matrix obtained in step a).
c) cellularization and/or loading with an active ingredient and induction of shape memory.

According to the present invention, a copolymer refers to a polymer consisting of repeated blocks of two or more homopolymers, in defined ratios, whereas physical mixture refers to the mixture of two or more polymers, in different ratios, which keep their structure unaltered.

The electrospinning instrument can be set with the process parameters indicated in the following Table 1:

TABLE 1

| Process parameters | Range | |
|---|---|---|
| Voltage | 10-+100 | kV |
| Flow-rate | 0.1-60 ml/hour | |
| | (1 needle) | |
| Needle-collector distance | 20-150 | mm |
| Spinneret Movement | 0-100 | mm |
| Spinneret speed | 0-300 | mm/s |
| Electrospinning time | 1-120 | min |

The sanitation step b) preferably takes place by immersing the matrices in a 70% v/v ethanol bath for 15-30 minutes and treatment with ultraviolet rays under a UV lamp for 12 hours (indicative and variable time). Sanitation is a treatment for reducing the microbial load that can be used in the preliminary study and development phases of the prototype. Sterilization is a validated treatment for reducing the microbial load and is compulsory for the finished product and for its application in vivo.

Alternatively, the matrices can be sterilized by irradiation with ionizing radiation, for example gamma-rays, or by plasma treatment.

After sanitation and/or sterilization treatment, the polymeric matrices are engineered with the cells (so-called cellularization according to the present invention).

According to a preferred embodiment, said step for cellularization and induction of the shape memory, c) comprises the following steps:
i) incubation of the polymeric matrix in a culture medium comprising cells at a temperature higher than the Tg of the copolymer or the physical mixture of the polymers used, the latter being characterized by a glass transition temperature ranging from 30° C. to 45° C.;
ii) rolling of the polymer chains around a sterile bar and exposure of the cellularized surface internally to a temperature lower than that of the Tg of the copolymer or of the physical mixture of polymers used, compatible with cell survival;
iii) immersion of the polymeric matrices with a stable cylindrical shape obtained in step ii) in a culture medium at a temperature equal to or higher than the Tg of the copolymer or of the physical mixture of polymers used for obtaining the unrolling of the polymeric matrices.

In a preferred embodiment of the cellularization step (when the PLA: PCL copolymer is characterized by a weight ratio of 70:30) the temperature of step i) is about 40° C.; the temperature of step ii) is about 5° C. and the temperature of step iii) ranges from 35 to 38° C., preferably 37° C.

In the step for producing the polymeric matrices, their cellularization and shape-memory induction, the process parameters indicated in Table 2 reported in the following Example 1 are preferably used.

The cells that are seeded on the polymer matrix in the above-mentioned step i) are selected from the group consisting of stem cells, mesenchymal cells, fat cells, umbilical cord cells, fibroblasts, endothelial cells, nerve cells, muscle cells, hepatocytes, intestinal cells, liver cells, lymphocytes, immune system cells, corneocytes and osteoblasts. In a preferred embodiment, human mesenchymal stem cells were used.

According to an alternative embodiment, the shape-memory polymeric matrix can further comprise one or more polymers having a glass transition temperature ranging from 30° C. to 45° C. selected from:
i) synthetic polymers selected from the group consisting of polyesters, polyacrylic derivatives, polyurethanes and their derivatives; and/or
ii) natural polymers selected from the group consisting of polysaccharides, hyaluronic acid, glycosaminoglycans and their derivatives. According to an alternative embodiment, the polymeric matrix is not cellularized but loaded with an active ingredient. This can be effected by direct bonding and functionalization of the polymeric matrix itself or by indirect bonding through spacers. The above-mentioned active ingredient can be of a chemical origin (i.e. a drug) or natural, in particular it can be chosen from the group that consists of non-steroidal or steroidal anti-inflammatory drugs, antibiotics, antineoplastics, growth factors (VEGF, FGF, EGF), nucleic acids (DNA, RNA or their fragments), hormones (e.g. glucagon or insulin), cytokines, chemokines, antibodies (or their fragments), products of a biotechnological origin and metals.

According to a preferred embodiment of the invention, the shape-memory polymeric matrix for local applications of cellular or pharmacological therapies can incorporate lipid and/or polymer and/or metal-based nanoparticulate systems carrying active ingredients with a pharmacological activity or having an intrinsic pharmacological activity.

Alternatively to the electrospinning of step a), the polymer matrix of the invention can be obtained by means of 3D bioprinting techniques, rotary jet spinning, solidification by solvent casting, in particular when the polymer or the mixture of PLA: PCL polymers does not have characteristics suitable for being electrospun or if a geometry that cannot be obtained with electrospinning is required.

The invention also relates to the cellularized shape-memory polymer matrix as described above for use in the medical field as a matrix in tissue regeneration or for therapy at the cellular level.

The present invention further relates to the use of the shape-memory polymeric matrix which incorporates an active ingredient as an in situ release system, wherein said active ingredient is selected from the group consisting of non-steroidal or steroidal anti-inflammatory drugs, antibiotics, antineoplastics, growth factors, nucleic acids, hormones, cytokines, chemokines, antibodies, nanoparticles, extravellular vesicles, liposomes and metals.

The present invention will now be described for illustrative but non-limiting purposes, according to a preferred embodiment with particular reference to the attached figures, in which:

FIG. 1 shows the steps of the shape-memory induction protocol for engineered polymer matrices. a) Original extended configuration (C1); b) Engineered polymer matrix rolled around a metal bar; c) Rolled configuration (C2); d) Unrolled configuration (C3).

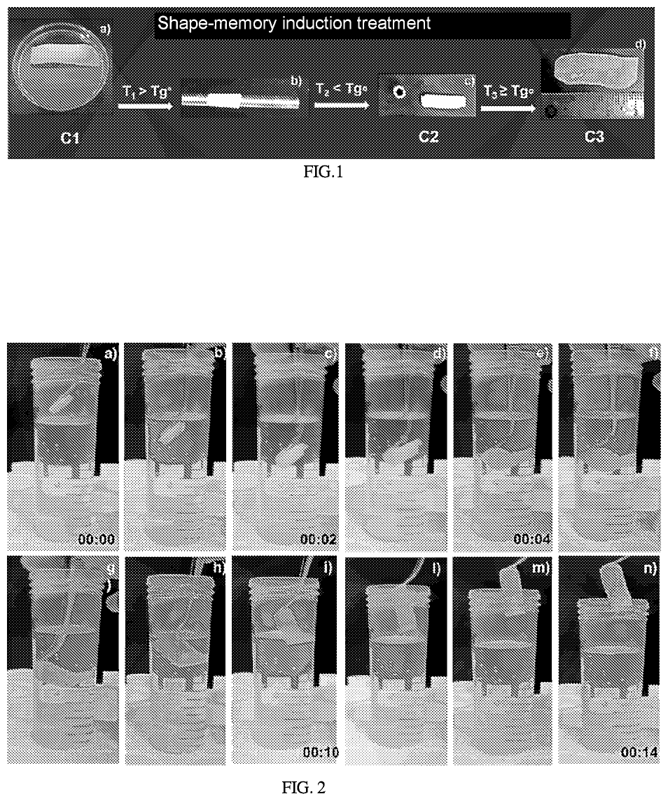

FIG. 2 shows the sequential images of the unrolling process of the engineered polymer matrix (passage from C2 to C3).

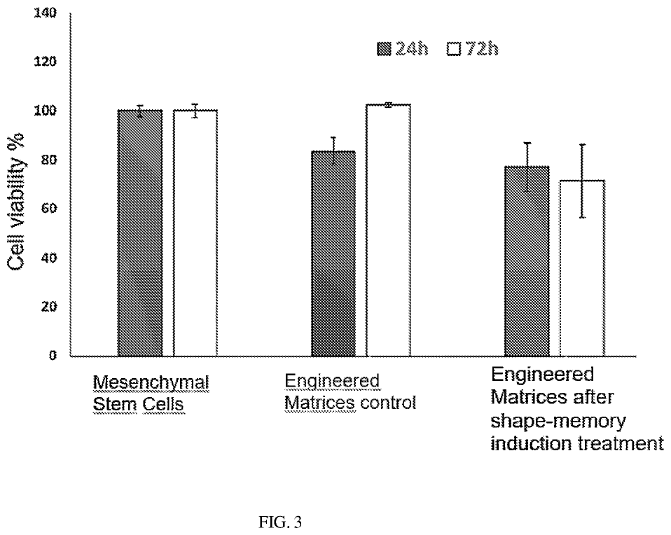

FIG. 3 shows the results of the cell viability assay evaluated on control mesenchymal stem cells, engineered matrices not subjected to shape-memory induction protocol (control) and engineered matrices after shape-memory induction treatment. The vitality test was performed after 7 days of cellularization plus 24 h (total 8 days) and 72 h (total 10 days) after shape-memory induction treatment.

FIG. 4 shows the nuclear staining images of a) control cells after 8 days and d) of cells adhered to the polymeric matrix after 7 days of cellularization plus 24 h after shape-memory induction treatment. b) Live and c) Dead, cell viability test on control cells after 8 days; e) Live and f) Dead, test on cells adhered to the polymer matrix after 7 days of cellularization plus 24 hours after shape-memory induction treatment.

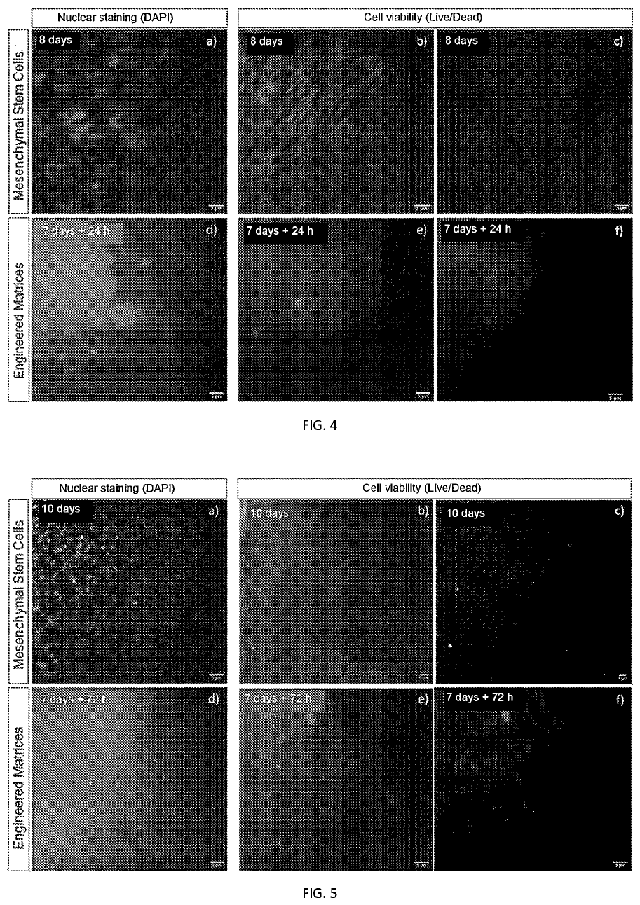

FIG. 5 shows the nuclear staining images of a) control cells after 10 days and d) of cells adhered to the polymeric matrix after 7 days of cellularization plus 72 h after shape-memory induction treatment. b) Live and c) Dead, cell viability test on control cells after 10 days; e) Live and f) Dead, test on cells adhered to the polymer matrix after 7 days of cellularization plus 72 hours after shape-memory induction treatment.

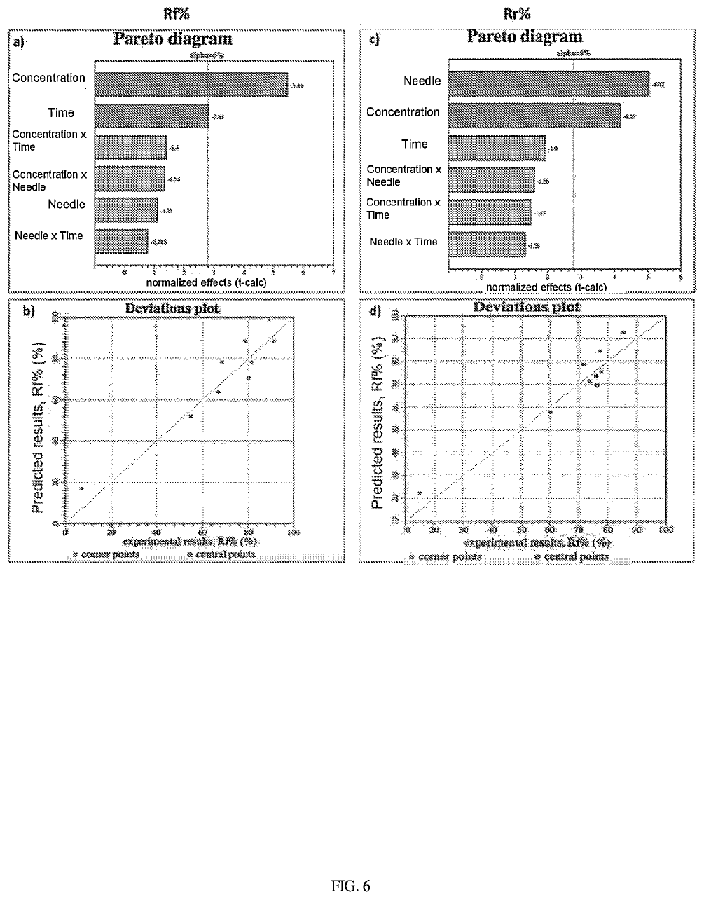

FIG. 6 shows in panel a) the Pareto diagram for the dependent variable Rf % (y1) (p-value <0.0001); in panel b) the deviation graph Rf % (y1) dependent variable (R2: 87.58%); in panel c) the Pareto diagram for the dependent variable Rr % (y2) (p value <0.0001); in panel d) the dependent variable of the deviation graph Rr % (y2) (R2: 89.89%).

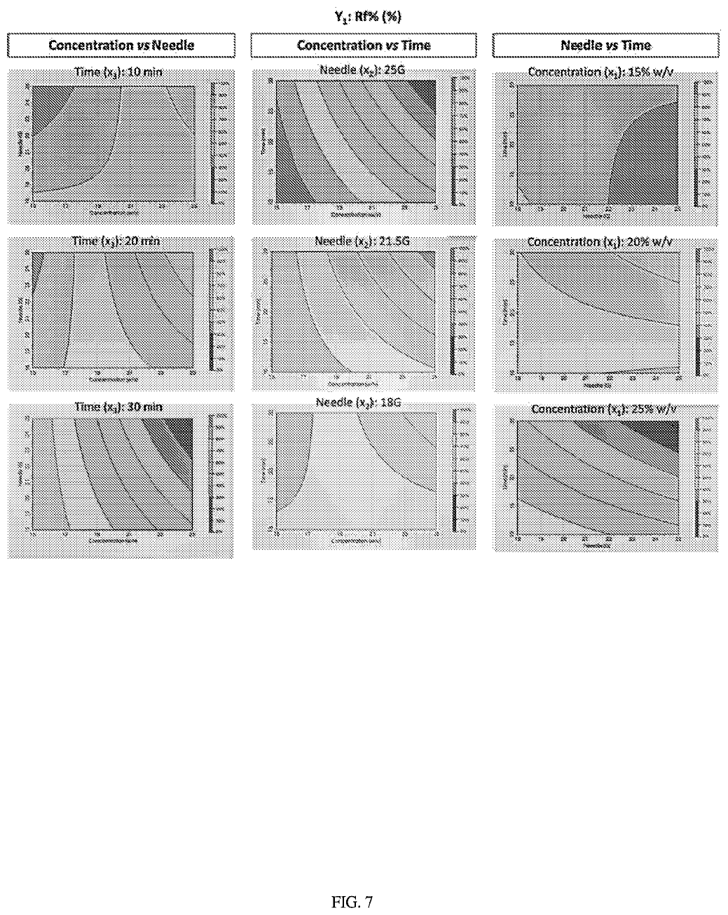

FIG. 7 shows the contour diagram for the dependent variable Rf %.

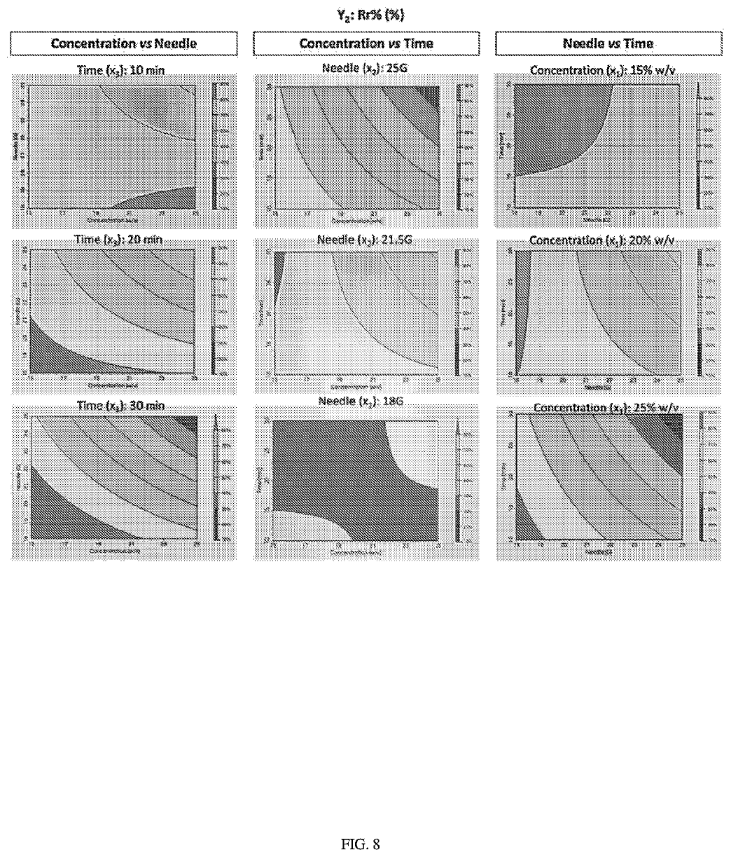

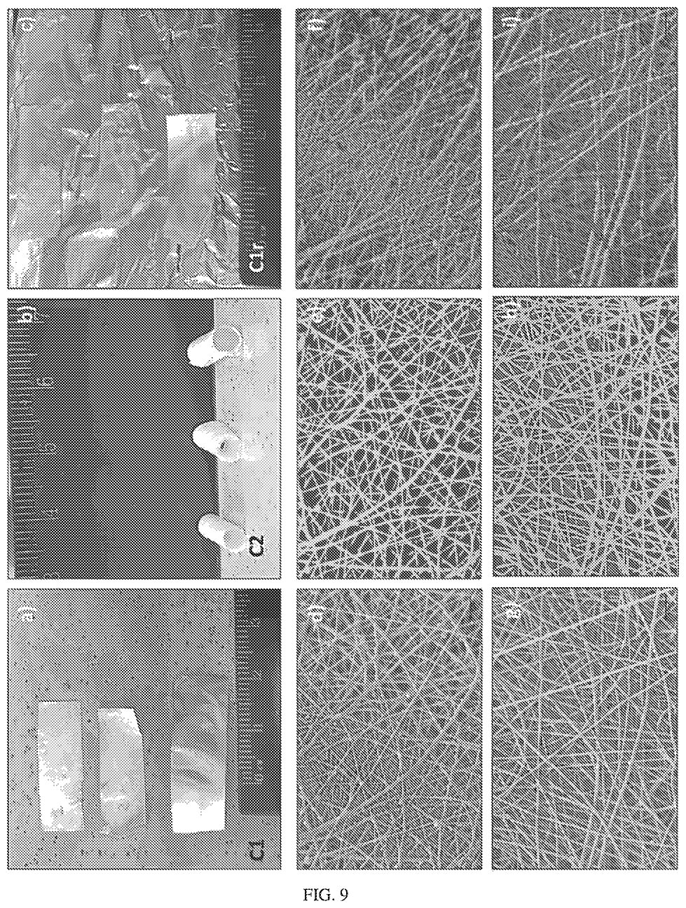

FIG. 8 shows the contour diagram for the dependent variable Rr %.

FIG. 9 shows in the upper panel, images a-c: shape-memory treatment. Image a) original configuration C1 of the electrospun tissue; image b) after the first heating treatment (T1>Tg°) and cooling bath (T2<Tg°) the electrospun tissues maintained their rolled shape (configuration C2) and image c) after treatment at a temperature close to Tg° (37° C.) shape-memory electrospun tissues recovered their original shape (C1r configuration). In the lower panel, images d-i: morphological characterization of electrospun PLA: PCL nanofibers obtained from polymer solutions at 15% w/v and using a 22 G needle for a deposition time of 20 minutes after shape-memory treatment. Image d) SEM of non-irradiated fibers; image e) ImageJ processing, fiber porosity analysis; image f) Image J processing, fiber orientation analysis; image g) SEM of gamma-irradiated fibers; image h) ImageJ processing, analysis of the porosity of gamma irradiated fibers; image i) ImageJ processing, analysis of the orientation of gamma-irradiated fibers.

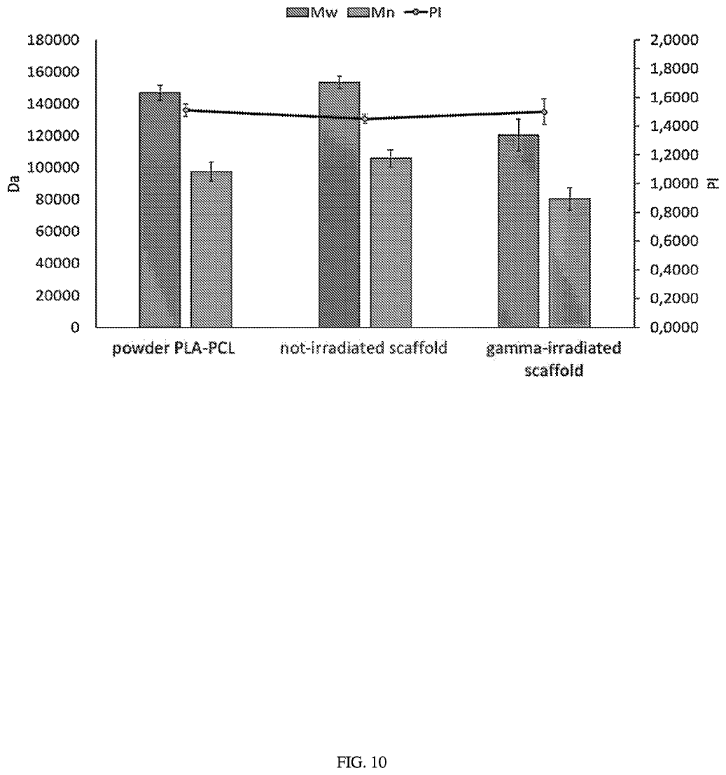

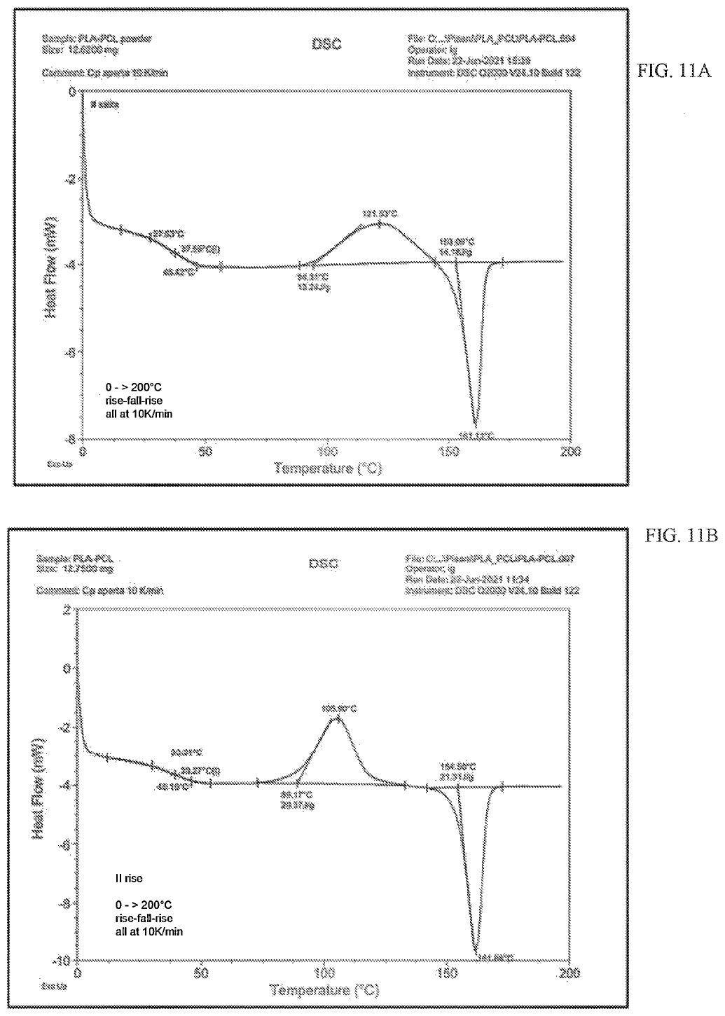

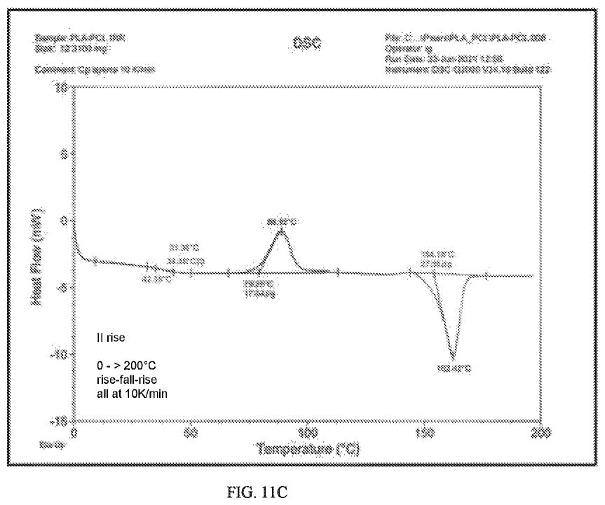

FIG. 10 shows the results of GPC analyses indicating Mw, Mn and PI of PLA-PCL powder, PLA-PCL tissue non-irradiated and irradiated with gamma rays.

FIG. 11 shows the results of the DSC analysis effected on a) PLA: PCL powder; b) electrospun tissues based on non-irradiated PLA: PCL and c) electrospun tissues based on gamma-irradiated PLA: PCL.

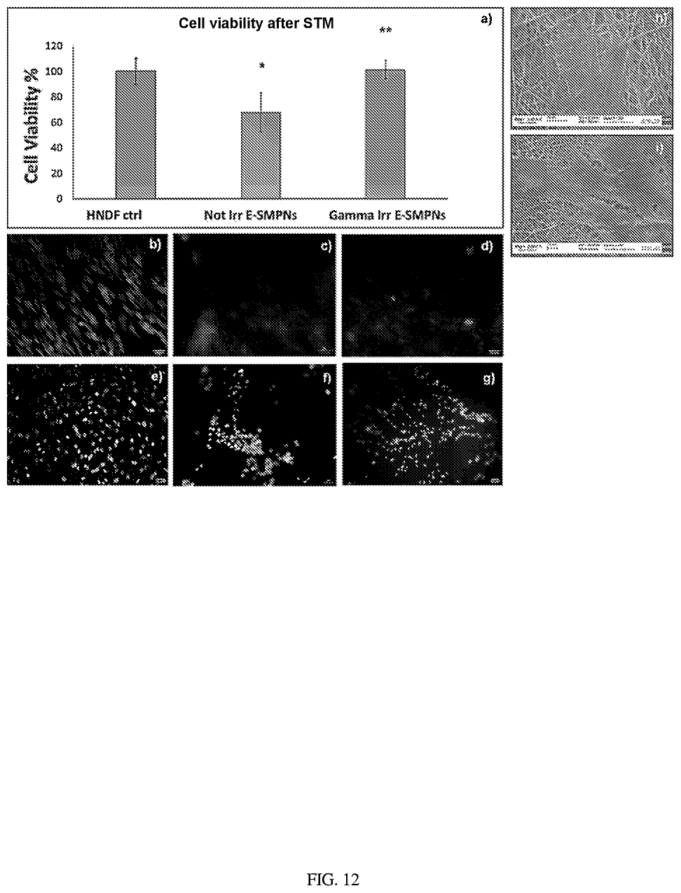

FIG. 12 shows in panel a) the results of the cell viability % determined by MTT assay on HNDF, incubated with non-irradiated PLA: PCL electrospun tissues and irradiated with gamma rays after 7 days of incubation and 6 hours after shape-memory treatment. Merge staining of Live (green)/Dead (red) staining of human skin fibroblast after 7 days of incubation; in panel b) control; in panel c) the non-gamma-irradiated engineered shape-memory tissue after-shape memory treatment; in panel d) engineered shape-memory tissue irradiated with gamma rays after shape-memory treatment. DAPI staining of NHDF after 7 days of incubation; in panel e) control; in panel f) the engineered shape-memory tissue non-irradiated with gamma rays; in panel g) the engineered shape-memory tissue irradiated with gamma rays. In panel h) SEM images of human skin fibroblast after 7 days of incubation of non-gamma-irradiated engineered shape-memory tissue and in panel i) of gamma-ray irradiated engineered shape-memory tissue.

The following illustrative but non-limiting examples of the invention are provided for a better illustration of the invention.

### EXAMPLE 1

### Method for Preparing the Engineered Shape-Memory Polymer Matrices According to the Invention

The polymer matrices of the invention are produced using a PLA-PCL copolymer in a 70:30 ratio.

The copolymer is solubilized in a concentration ranging from 10 to 30% w/v, preferably 20% w/v, in a mixture of organic solvents such as dichloromethane and dimethylformamide in a ratio ranging from 80:20 to 60:40, preferably 70:30.

The following process parameters were applied for the electrospinning: Voltage (30 kV), flow (0.5 ml/h), needle-collector distance (150 mm), spinneret movement (70 mm), spinneret speed (70 mm/sec), electrospinning time (20 min).

The matrices obtained were cut to the most suitable size for the cellularization and shape-memory induction treatment.

Once cut into a rectangular shape (for example 3.0×1.5×0.007 cm), the polymeric matrices can be sanitized by immersing them in a 70% v/v ethanol bath for 15-30 minutes and with ultraviolet treatment under a UV lamp for 12 hours.

The matrices can be sterilized by irradiation with ionizing radiation, for example gamma-rays at a dose of 25 kGray (or lower depending on the use).

After sanitation and/or sterilization treatment, the polymeric matrices are cellularized. In this specific example, human mesenchymal stem cells were used.

The cellularization of the matrices in static cell cultures was carried out for 7 days in order to ensure adequate adhesion of the cells on the polymeric support and to have an initial proliferation (the cells can also be sown in an appropriate number to guarantee the desired action and left in contact with the matrix for the time necessary for their adhesion e.g. 4-6 h and subsequently treated in order to induce shape memory). At the end of the cellularization, the engineered matrices are subjected to the first step of the shape-memory treatment in order to induce a rolled shape to the cellularized matrices.

The engineered matrices are incubated in a suitable culture medium (DMEM and/or PBS pH 7.4) at a temperature higher than the Tg of the copolymer used, preferably 40° C., and after a time suitable for guaranteeing the softening of the polymer chains (in this example 10 min), they are extracted from the culture system and manually rolled around a sterile steel bar having selected dimensions (in this example 2 mm) in order to expose the cellularized surface internally.

The engineered matrices are incubated in a suitable culture medium (DMEM and/or PBS pH 7.4) at a temperature higher than the Tg of the copolymer used, preferably 40° C., and after a time suitable for guaranteeing the softening of the polymer chains (in this example 10 min), they are extracted from the culture system and manually rolled around a sterile steel bar having selected dimensions (in this example 2 mm) in order to expose the cellularized surface internally.

The matrices rolled around the bar are immersed in a medium compatible with cell survival (DMEM and/or PBS pH 7.4) and brought to a temperature below that of the polymer Tg but compatible with cell survival (5° C.).

The matrices are kept at temperatures below Tg for a time suitable for allowing the engineered matrix to acquire a stable rolled cylindrical shape, preferably 10 min.

The rolled matrices carrying the cells in the inner portion are brought back to temperatures close to/higher than those of the polymer Tg by immersing them in a culture medium (DMEM and/or PBS pH 7.4) ranging from 35 to 38° C., preferably 37° C. The return to temperatures above the Tg of the polymer and suitable for cell survival allows the spontaneous unrolling of the engineered matrices in reduced times in a few minutes (in this example 10-14 sec, see FIG. 2).

The matrices subjected to the external stimulus of temperature variation are capable of recovering their original extended configuration and at the same time ensure the maintenance of suitable cellular vitality.

Table 2 hereunder summarizes the values of the process parameters and the relative ranges within which to move in order to obtain engineered shape-memory matrices capable of ensuring optimal rolling/unrolling and suitable cell viability.

TABLE 2

| Parameters | Range | |
|---|---|---|
| Concentration of PLA-PCL polymer solution | 10-30% | w/v |
| Solvents | Dichloromethane | |
| | Dimethylformamide | |
| | Acetone | |
| | Chloroform | |
| | In all ratios | |
| Form of electrospun matrices | Square, rectangular, | |
| | and/or round | |
| Number of seeded cells | 5 × 10³-1 × 10⁶ | cells |
| Shape-memory Induction temperature | 0-50° | C. |
| Shape-memory induction time | 1-60 | min |
| Bar size for rolling | 0.5-20 | mm |
| Medium in contact with engineered matrices | DMEM, PBS (pH 7.4), | |
| | HEPES (pH 7.4) | |
| Cellularization time of matrices | 3-21 | days |

The viability of the cells seeded and proliferated on the polymer matrices was verified before and after being subjected to the protocol for shape memory. The MTT cell viability assay was performed after 7 days of cellularization plus 24 h after shape-memory treatment (for a total of 8 days overall) and after 7 days of cellularization plus 72 h after shape-memory treatment (for a total of 10 days in total). The results are shown in FIG. 3.

The presence of cells on the engineered matrices after the shape-memory process was confirmed by nuclear staining (DAPI) and cell viability tests (Live/Dead). FIGS. 4 and 5 show the images obtained by a fluorescence microscope of the engineered matrices for 7 days and 24 h after shape-memory treatment (for a total of 8 days overall) and after 7 days plus 72 h from shape-memory treatment (for a total of 10 days in total).

### EXAMPLE 2

### Study on the Interaction Between the Process Variables Relating to the Production of the Engineered Shape-Memory Tissue According to the Invention

A statistical approach called “Design of Experiment” (DOE) was used for defining the interaction between the process vaibles relating to the production of the engineered shape-memory tissue.

The following were defined as independent variables (x):
the concentration of polymeric solution (w/v %);
spinning time (min)
the opening diameter of the needle hole (Gauge—G).

Their reciprocal influence was then defined on the dependent variables (y) defined as: Rf % (capacity of the shape-memory tissue of maintaining its induced temporary shape) and Rr % (capacity of the shape-memory tissue of recovering its original form).

A mathematical model was obtained from the DOE useful for predicting the Rf % and Rr % values of the memory tissues produced with the poly-L-lactide-poly-ϵ-caprolactone copolymer (PLA: PCL) 70:30.

As an experimental test for the validation of the mathematical model, a shape-memory tissue was produced with PLA: PCL 70:30 starting from its solution at 15% w/v in CH₂Cl₂ (MC)/dimethylformamide (DMF) 70:30% v/v ratio, 22 G needle and deposition time of 20 minutes. Morphological (SEM), physico-chemical (GPC and DSC), mechanical (uniaxial tensile tests) and biological (viability and cell adhesion) characterizations were subsequently effected.

Polymer solutions were prepared at different concentrations (15% w/v, 20% w/v and 25% w/v) using mixtures of solvents with an MC and DMF ratio of 70:30.

The parameters of the electrospinning process were set as follows: voltage (30 kV), flow-rate (0.5 ml/h), needle-collector distance (15 cm). A flat metal collector was used for collecting the electrospun fibers. The polymeric solutions were electrospun keeping the temperature (25° C.±3° C.) and relative humidity (30±4%) values constant. Different sized needles (18 G, 22 G and 25 G) and different deposition times (10, 20 and 30 min) were used for obtaining the electrospun tissues, as obtained from the DOE and indicated in Table 3 hereunder.

The electrospun tissues obtained were cut into a rectangular shape (2.5×1.5 cm) and were subjected to shape-memory treatment and the Rf % and Rr % data were calculated with equations 1 and 2 (Eq1 and Eq2), respectively:

$$\\ \begin{matrix}Rf ( \% ) = 1 - [ ( {ε}_{f} - {ε}_{i} ) / ε f ] \times 100 & ( Eq . 1 )\end{matrix}$$

$$\\ \begin{matrix}Rr ( \% ) = ( {ε}_{p} / {ε}_{0} ) \times 100 & ( Eq . 2 )\end{matrix}$$

wherein ϵ_{f} is the measurement of the rolled tissue after the cold treatment; ϵᵢ is the reference measurement of the bar used as a mould for the rolling and induction of the temporary form; ϵₚ indicates the size of the tissue which has recovered its original size; ϵ₀ is the original size of the tissue before shape-memory treatment used as a reference.

The Rf % and Rr % data were further processed with DOE to find correlations and obtain the mathematical model.

Each analysis was performed in triplicate using a digital caliper and ImageJ software for the measurements, and the data are indicated in Table 3 as Rf (%) and Rr (%) values with standard deviation (±SD).

TABLE 3
DOE indicating for each line the value of independent
variables (x) used for each type of test and the
values of dependent variables (y) obtained

| | x | | | | |
|---|---|---|---|---|---|
| Test | Concentra- | Needle | Time | y | |
| number | tion(w/v) | (G) | (min) | Rf % (%) | Rr % (%) |
| 1 | 15 | 18 | 10 | 81.35 ± 3.4 | 77.87 ± 2.5 |
| 2 | 25 | 18 | 10 | 68.54 ± 4.2 | 77.34 ± 3.1 |
| 3 | 15 | 25 | 10 | 89.04 ± 3.5 | 71.50 ± 4.6 |
| 4 | 25 | 25 | 10 | 66.89 ± 3.3 | 60.18 ± 3.6 |
| 5 | 15 | 18 | 30 | 78.62 ± 4.9 | 85.52 ± 2.9 |
| 6 | 25 | 18 | 30 | 55 ± 3.1 | 76.04 ± 4.1 |
| 7 | 15 | 25 | 30 | 91.47 ± 5.6 | 73.73 ± 2.5 |
| 8 | 25 | 25 | 30 | 7.11 ± 1.3 | 15.03 ± 5.3 |
| 9-10-11 | 20 | 22 | 20 | 80.01 ± 2.5 | 76.34 ± 3.2 |

Table 3 shows that the 15% w/v and 20% w/v solutions have higher Rf % values, whereas the solutions obtained using the 18 G needle appear to have better Rr % values.

The shape-memory tissues obtained with higher concentration solutions (25% w/v) did not guarantee an adequate shape-memory effect.

The data obtained made it possible to identify the correlation of the independent variables (x) that most influence the desired outputs for Rf %.

The Pareto diagrams shown in FIG. 6, panel a) show the absolute normalized effects (t-values) sorted by size. Factors with an absolute t value greater than a tabulated t value for a given significance level (5%) and degree of freedom (shown as a vertical line) were found to be statistically significant. In this case, the concentration of the polymer solution (w/v %) and the electrospinning time (min), are the two parameters that most influence the Rf %.

The deviation graph shown in FIG. 6, panel b) visually compares the experimental results of the Rf % obtained and the results predicted by the DOE model, for each sample performed. The graph shows which result the model (vertical axis) would predict for each experimental result (horizontal axis). The points closest to the diagonal line of the graph are the most reliable in the model, at least within the range of values studied. For the Rf % value, a determination coefficient (R²) of 87.58% was determined.

The data obtained made it possible to identify the correlation of the independent variables (x) that most influence the desired outputs for Rr %. The Pareto diagrams shown in FIG. 6, panel c) show the absolute normalized effects (t-values) sorted by size. In this case, for the parameter Rr %, the size of the needle (G) and the concentration of the polymer solution (w/v %) are the two most influencing parameters. The deviation graph shown in FIG. 6, panel d) visually compares the experimental results of Rr % obtained and the results predicted by the DOE model, for each sample performed. A determination coefficient (R²) for Rr % of 89.89% was obtained.

The data were also presented using a colour contour diagram, a two-dimensional view of the dependence of the response variables Rf % and Rr % (FIGS. 7-8), and two independent variables.

Using the contour diagrams, it is possible to visualize the Rf % and Rr % capacity of the shape-memory tissues obtained from the desired PLA: PCL solution in different concentration ranges from 15% w/v to 25% w/v, needle from 18 G to 25 G at different deposition times (10, 20 and 30 minutes).

A mathematical model was obtained from the DOE project, capable of calculating theoretical Rf % and Rr % for specific PLA-PCL concentration values, needle diameter and deposition time used.

The predictive equations obtained for the value of Rf % (Eq. 3) and Rr % (Eq. 4) are indicated hereunder.

$$\\ \begin{matrix}Rf \% = - 168.5 + 10.839786 Concentration ( p / v \% ) + 11.91 Needle diameter ( G ) + 5.8851071 Time ( min ) - 0.50057143 Concentration ( p / v \% ) \times Needle diameter ( G ) - 0.185255 Concentration ( p / v \% ) \times Time ( min ) - 0.14671429 Needle diameter ( G ) \times Time ( min ) & ( Eq . 3 )\end{matrix}$$

$$\\ \begin{matrix}Rr \% = 123.5 + 10.031571 Concentration ( w / v \% ) + 8.6517857 Needle diameter ( G ) + 6.1426071 Time ( min ) - 0.42864286 Concentration ( p / v \% ) \times Needle diameter ( G ) - 0.140825 Concentration ( w / v \% ) \times Time ( min ) - 0.17596429 Needle diameter ( G ) \times Time ( min ) & ( Eq . 4 )\end{matrix}$$

A composition of PLA was selected as the most suitable combination of parameters: PCL at 15% w/v, needle 22 G and deposition time of 20 minutes.

Using the mathematical equations Eq.3 and Eq.4 theoretical values of Rf %=89.31% and Rr %=79.04% were obtained. A subsequent characterization was effected on PLA: PCL-based shape-memory tissues prepared using the electrospinning process parameters used above and the experimental data Rf % and Rr % were evaluated (Table 3). Furthermore, the electrospun samples were cut into a rectangular shape (2.5×1.5 cm) and placed in sterile tubes inert to gamma radiation for subsequent gamma radiation treatment (25 kGy). Sterilization was effected at controlled room temperature (25° C.±3° C.). After gamma irradiation, the electrospun tissues were subjected to shape-memory treatment and their shape-memory properties were verified and compared with non-gamma irradiated electrospun tissues.

The Rf % and Rr % values were evaluated using equations 1 and 2. Analyses were carried out in triplicate and the data reported as mean±SD data (Table 4).

TABLE 4
Experimental Rf % and Rr % values for PLA:PCL shape-
memory tissues non-irradiated and gamma-irradiated

| Sample | Rf % | Rr % |
|---|---|---|
| Non-irradiated shape-memory tissue | 88.21 ± 3.4% | 81.12 ± 5.3% |
| Gamma-irradiated shape-memory tissue | 93.21 ± 2.7% | 86.12 ± 3.3% |

Morphological, physico-chemical, mechanical and biological characterizations were performed on non-irradiated and irradiated shape-memory tissues (15% w/v, 22 G needle and 20 min deposition time).

Morphological characterization on non-irradiated and irradiated samples after shape-memory treatment was performed by SEM. The images obtained were processed using the ImageJ software for evaluating the size and porosity of the fibers (FIG. 9).

Gel Permeation chromatography (GPC) was effected for evaluating the molecular weight (Mw) and the molecular number (Mn) of the PLA: PCL polymer constituting the shape-memory tissues, before and after gamma irradiation. The results (FIG. 10) confirmed that the polymer constituting the electrospun tissues irradiated with gamma rays underwent a Mw reduction of about 21.53% and a Mn reduction of about 17.55% after irradiation of a dose of 25 kGy. Considering that the sensitivity of the GPC analysis is within a 10% variation, the reduction in the Mw and Mn after irradiation is significant. Surprisingly, the PI did not change significantly after irradiation (1.45 PI for non-irradiated and 1.5 PI for gamma irradiation). The standard deviation PI (1.5±0.09), however, is greater in irradiated samples.

The PLA: PCL copolymer purchased indicates Tg range values of 32-42° C. in its data sheet. DSC analyses confirmed that the Tg value of the PLA:PCL powder is included in the tabulated range values (Tg=37.59° C.) (FIG. 11, panel a). The non-irradiated electrospun tissue showed a Tg=39.27° C. (FIG. 11, panel b), confirming that the electrospinning process affects the thermal behaviour of the copolymer as it influences the solid state of the polymer in terms of crystallinity and amorphism percentage. The gamma-irradiated electrospun tissues, on the other hand, showed a lower Tg value=34.86° C. (FIG. 11, panel c).

The axial tensile test was performed on non-irradiated and gamma-irradiated PLA: PCL-based tissues. As indicated after the physico-chemical characterization, the gamma irradiation caused a decrease in the Mw, Mn and Tg values. Mechanical characterization showed a significant reduction in elasticity for gamma-irradiated tissues, as indicated in Table 5 hereunder.

TABLE 5
Young's modulus and ultimate elongation % of non-irradiated and
gamma-irradiated electrospun tissues based on PLA:PCL ultimate

| | | Maximum ultimate |
|---|---|---|
| | Young's Modulus | elongation |
| Sample | (MPa) | (%) |
| Non-irradiated | 0.33 ± 0.03 | 200 ± 12.5 |
| Gamma-irradiated | 0.71 ± 0.12 | 160 ± 15.6 |

The biological characterization, performed on non-irradiated and irradiated electrospun tissues, engineered with human dermal fibroblasts (HNDF) and subjected to shape-memory treatment comprises:
% viability assay (MTT) (FIG. 12, panel a);
colorimetric viability assay (live/dead) (FIG. 12, panels b, c, d);
nuclear fixation of cells (DAPI) (FIG. 12, panels e, f, g);
morphological analysis (SEM) (FIG. 12, panels h, i).

### BIBLIOGRAFY

[1] R. Tatu, M. Oria, S. Pulliam, L. Signey, M. B. Rao, J. L. Peiro, C. Y. Lin, Journal of biomedical materials research. Part B, Applied biomaterials 107 (2) (2019) 295-305.
[2] M. Bao, X. Lou, Q. Zhou, W. Dong, H. Yuan, Y. Zhang, ACS applied materials & interfaces 6 (4) (2014) 2611-2621.
[3] Q. Zhao, J. Wang, H. Cui, H. Chen, Y. Wang, X. Du, Adv Funct Mater 28 (29) (2018) 1801027.
[4] L. -F. Tseng, P. T. Mather, J. H. Henderson, Acta Biomaterials 9 (11) (2013) 8790-8801.

1. A method for producing a shape-memory polymeric matrix comprising:
a) electrospinning of a PLA-PLC copolymer or PLA and PLC polymers in a physical ixture wherein the PLA: PCL weight ratio ranges from 80:20 to 60;
b) sanitizing or sterilizing the polymer matrix obtained in step a); and
c) cellularizing and/or loading with an active ingredient.

2. The method according to claim 1, wherein the PLA: PCL weight ratio is 70:30.

3. The method according to claim 1, wherein said step c) comprises:
i) incubating the polymeric matrix in a culture medium comprising cells at a temperature higher than the Tg of the copolymer or the physical mixture of polymers used;
ii) rolling the polymer chains around a sterile bar so as to expose the cellularized surface internally to a temperature lower than that of the Tg of the copolymer or the physical mixture of polymers used, compatible with cell survival, to form polymeric matrices having a stable cylindrical shape;
iii) immersing the polymeric matrices having a stable cylindrical shape obtained in step ii) in a culture medium at a temperature equal to or higher than the Tg of the copolymer or of the physical mixture of polymers used for obtaining the unrolling of the polymeric matrices.

4. The method according to claim 1, wherein said cells of step i) are selected from the group consisting of stem cells, mesenchymal cells, adipose cells, umbilical cord cells, fibroblasts, endothelial cells, nerve cells, muscle cells, hepatocytes, intestinal cells, liver cells, lymphocytes, immune system cells, corneocytes and osteoblasts.

5. The method according to claim 1, further comprising adding one or more polymers having a glass transition temperature ranging from 30° C. to 45° C. selected from:
i) synthetic polymers selected from the group consisting of polyesters, polyacrylic derivatives, polyurethanes and their derivatives; and/or
ii) natural polymers selected from the group consisting of polysaccharides, hyaluronic acid, glycosaminoglycans and their derivatives.

6. The method according to claim 1, wherein said active ingredient of step c) is bound within the polymeric matrix by direct bonding and functionalization of the same or by indirect bonding through spacers.

7. The method according to claim 1, wherein said active ingredient of step c) is selected from the group consisting of non-steroidal or steroidal anti-inflammatory drugs, antibiotics, antineoplastics, growth factors, nucleic acids, hormones, cytokines, chemokines, antibodies, nanoparticles, products of a biotechnological origin, liposomes, metals and combinations thereof.

8. The method according to claim 1, produced alternatively to the electrospinning of step a) by 3D printing bioprinting, rotary jet spinning solidification by evaporation of the solvent.

9. The method according to claim 1, further comprising adapting the cellularized shape-memory polymeric matrix for use in the medical field as a matrix in tissue regeneration or for therapy at the cellular level.

10. The method of claim 1 further comprising incorporating an active ingredient as an in situ release system, wherein said active ingredient is selected from the group consisting of non-steroidal or steroidal anti-inflammatory drugs, antibiotics, antineoplastics, growth factors, nucleic acids, hormones, cytokines, chemokines, antibodies, nanoparticles, products of biotechnological origin, liposomes, metals and combinations thereof.
